(19) United States
(12) Patent Application Publication
(10) Pub. No.: US 2014/0121493 A1
(43) Pub. Date: May 1, 2014

(54) System and Apparatus for Multimodality-compatible High-quality Intravital Radionuclide Imaging

(71) Applicants: Kuangyu Shi, Munich (DE); Zhen Liu, Munich (DE); Sibylle I. Ziegler, Munich (DE); Markus Schwaiger, Munich (DE)

(72) Inventors: Kuangyu Shi, Munich (DE); Zhen Liu, Munich (DE); Sibylle I. Ziegler, Munich (DE); Markus Schwaiger, Munich (DE)

(73) Assignee: Kuangyu Shi, Munich (DE)

(21) Appl. No.: 13/666,127

(22) Filed: Nov. 1, 2012

(51) Int. Cl. A61B 6/00; A61M 16/01; A61B 6/03; A61B 6/04; A61B 5/055; A61B 5/00

(52) U.S. Cl. CPC A61B 6/4417; A61B 5/0555; A61B 5/0035; A61B 6/037; A61B 6/0428; A61B 5/0071; A61B 5/0068; A61B 5/0042; A61B 6/501; A61B 6/508; A61B 6/481; A61B 5/4848; A61M 16/01
USPC 600/411

(57) ABSTRACT

The present invention discloses an imaging system and apparatus to obtain high-resolution low-noise intravital radionuclide imaging based on transparent window chamber. This imaging system is dedicated to preclinical research. It comprises a transparent window chamber, in particular a dorsal skin window chamber or a cranial chamber or an ear chamber or a spine cord chamber on a living animal, and a high-quality radionuclide imaging camera for the imaging of positron or electron. The apparatus is compatible with multimodality imaging, in particular magnetic resonance imaging (MRI), microscopy imaging including fluorescence microscopy, phosphorescence microscopy and two-photon microscopy.

The present invention discloses an imaging system and apparatus to obtain high-resolution low-noise intravital radionuclide imaging based on transparent window chamber. This imaging system is dedicated to preclinical research. It comprises a transparent window chamber, in particular a dorsal skin window chamber; cranial chamber, ear chamber or spine cord chamber on a living animal and a high-quality radionuclide imaging camera for the imaging of positron or electron. The apparatus is compatible with multimodality imaging, in particular magnetic resonance imaging (MRI), microscopy imaging including fluorescence microscopy, phosphorescence microscopy and two-photon microscopy.

The system of the present invention enables a reliable link between macroscopic imaging and microscopic physiological measurements. It allows high quality radionuclide imaging, high compatibility with multiple imaging modalities, precise co-registration of images obtained from different sources at different times and intact longitudinal multimodal observation. Thus the present invention can assist the validation and development of pharmaceutical, imaging, diagnostic and therapeutic techniques and strategies.

In contrast to conventional anatomical imaging such as CT or MRI, molecular imaging extends the clinical frontline to fundamental molecular pathways in organisms noninvasively, which supports the individualization of healthcare. Among all the molecular imaging modalities, radionuclide imaging such as positron emission tomography (PET) or single photon emission computed tomography (SPECT) are most widely used in clinical practice due to their high sensitivity of physiological differences and have shown enormous clinical value for example in early detection of cancer, staging, localization and therapy prognosis.

Radionuclide imaging is achieved through injection of radiolabeled molecular biomarkers, which generate contrast between normal and abnormal tissues according to their different metabolic properties of the injected biomarker. Various biomarkers have been developed for the detection of different physiological functions such as glycolysis (e.g. [18F]FDG, 99mTc-HMPAO), perfusion (e.g. [13N]Ammonia, 99mTc-tetrofosmin), hypoxia (e.g. [18F]Fmiso), proliferation (e.g. [18F]FLT) and so on.

The radioactive signals emitted from the radiolabeled biomarkers can be detected by radiation cameras (Scintigraphy), SPECT for single photon emitters or coincidence detectors (PET) for positron emitters.

After injection into the body, molecular biomarkers are delivered through macro- and microcirculatory system into tissues and then get either metabolized in the target area or cleared out. This complex procedure causes the acquired image to be influenced by many confounding factors, such as vascular delivery, interstitial transport and renal clearance. The interpretation of molecular imaging towards characteristics of the tumor microenvironment is therefore not straightforward.

For pharmaceutical development of new imaging biomarker or the application of clinical diagnosis and therapy planning, the assessment and validation of molecular imaging and its evaluation methods is necessary.

The assessment of molecular images requires a reliable link from macroscopic imaging to microscopic measurements. However, such a reliable link is not straightforward.

Conventionally, tumors need to be resected after animal scarification and be cut into sections for the investigation by microscopy. Although these in vitro methods have been widely used in various applications, they are destructive and have limited ability to provide insight into in vivo dynamics. The inconsistency between in vivo and in vitro does not meet the requirements for clinical applications such as biologically guided radiotherapy.

Furthermore, there exists a huge difference between typical preclinical and clinical molecular images (˜mm) and microscopic measurements (˜μm). The current preclinical PET can reach a resolution of approximately 1 mm while the preclinical SPECT can achieve 0.4 mm resolution for small animals. The measured signal in an imaging element is an integration of the signal over a relatively large scale of heterogeneous tumor microenvironment, which makes the validation of molecular imaging even more difficult.

The resolution of current radionuclide imaging devices is relatively low compared to morphological imaging modalities. For preclinical research of radionuclide imaging, high resolution is preferred.

Another problem for a reliable link from macro to micro is the co-registration of images. It is very hard to find useful landmarks or similarities between the images with such a huge resolution difference and typical co-registration algorithms are not applicable here. Also, the complicated preparation procedure of the histology sections introduces lots of distortions, thus it is almost impossible to localize the physiological features precisely in conventional imaging methods.

As the evolving of combined imaging modalities, such as PET/CT, PET/MRI, multimodal imaging is increasingly available in clinical practice. Corresponding multimodal quantitative analysis strategies is desired to assist the improvement of cancer diagnosis and therapy planning.

One major challenge for multimodality imaging is the co-registration between images obtained at different time, with different procedures and of different resolution.

From Cho, H., Ackerstaff, E., Carlin, S. et al.: “Noninvasive multimodality imaging of the tumor microenvironment: registered dynamic magnetic resonance imaging and positron emission tomography studies of a preclinical tumor model of tumor hypoxia. Neoplasia, 11 (2009) 247-59 it is known that fiducial markers can be inserted into tumor to assist the co-registration of MRI and PET images. In addition, immobilization foams need to be applied to the animal to fix the position and gesture. The procedure is destructive and not suitable for longitudinal observation. The registration error is still relatively large compared with the microscopic features.

Transparent window chamber is an effective tissue observation apparatus for intravital imaging. It sets up observable tissue microenvironment between or behind a fixed transparent window on an intact animal, which enables in-depth longitudinal observation of tissue physiologies.

Transparent window chamber can be generally classified into two categories based on tissue preparations: chronic window chamber and acute window chamber. Chronic window chamber allows continuous noninvasive, long-term monitoring of tissue pathologies. Typical examples are ear chamber, dorsal skinfold chamber, cranial chamber, hamster-cheek-pouch-window and spine cord chamber. Acute window chamber allows the observation of orthotopic tumors. However it does not support repeated or long-term observations. Typical acute window chamber includes hamster cheek pouch, mesentery, liver or pancreas chamber.

Intravital microscopy can be applied to transparent window chamber to obtain intact in vivo imaging of physiological features. In particular, the intravital microscopy includes fluorescence microscopy, phosphorescence lifetime imaging, confocal laser scanning microscopy and multi-photon microscopy.

Transparent window chamber has been applied in many studies of various purposes, such as microcirculation, angiogenesis, hypoxia and molecular dynamics and therapeutic interactions. In vivo tissue morphological and metabolic characteristics like acidity, oxygen tension can be obtained on from intravital imaging on transparent window chamber.

US 2004/0151666 A1 discloses a rodent mammary transparent window chamber for intravital microscopy of orthotopic breast cancer. It enables the investigation of the cellular behavior of implanted tumor cells in an orthotopic environment and to observe the earliest events during angiogenesis.

US 2004/0043462 A1 discloses a transparent window chamber for in vivo delivery of an active agent, which includes therapeutic and screening methods for in vivo screening of angiogenesis and/or tumor growth modulating agents.

US 2012/0035444 A1 discloses in vivo drug screening systems based on transparent window chamber.

Gaustad, J. V., Brurberg, K. G., Simonsen, T. G., et al.: “Tumor vascularity assessed by magnetic resonance imaging and intravital microscopy imaging.” Neoplasia, 10 (2008) 354-62 discloses a method to combine dynamic contrast enhanced MRI and intravital microscopy imaging and to validate the blood flow estimation of dynamic contrast enhanced MRI based on fluorescence measurement with intravital microscopy.

Although transparent window chamber has advantages for intravital imaging in revealing underlying tissue pathologies, it is in general not easy to directly apply them in the research of radionuclide imaging. One limitation for that purpose is the low resolution of current PET or SPECT. It is almost impossible to obtain a sufficient image of a surface tissue.

Most radiolabeled tracers of PET or SPECT emit positrons or electrons. Imaging positron or electrons directly has the potential to reach a resolution of microscopic level such autoradiography. However, positron or electron has limited penetration either in air or body. A direct imaging device of positron and electron needs to contact the imaging object as close as possible.

Liu, Z, Chen, L, Barber, S., et al.: “Direct positron and electron imaging of tumor metabolism and angiogenesis in a mouse dorsal skin window chamber model,” 59th Annual Meeting of the Society of Nuclear Medicine, Miami, Fla., June, 2012 discloses a high-resolution and high-sensitivity imaging system for direct imaging of positrons or electrons on transparent window chamber for in vivo studies of the tumor microenvironment. It needs an ultrathin phosphor film to be placed between the investigated tissue and a lens-coupled CCD camera to image the scintillation light excited by a positron/electron-emitting object. This method needs additional scintillation material between the camera and the tissue. Although the phosphor film can be fit into a conventional window chamber and enable the imaging of positron and electron within the observation window. However the imaging of the scintillation light from the phosphor film is easy to be influenced by noise.

From Russo, P., Lauria, A., Mettivier, G. et al.: “18F-FDG positron autoradiography with a particle counting silicon pixel detector.” Phys Med Biol, 53 (2008) 6227-43 and Mettivier, G., Montesi, M. C. and Russo, P.: “Digital autoradiography with a Medipix2 hybrid silicon pixel detector.” Nuclear Science, IEEE Transactions on, 52 (2005) 46-50 it is know that a high-quality positron or electron imaging can be achieved with silicon pixel detector, which can read the incident particle energy deposited in silicon detector directly using single pixel readout circuit. Thus it has high signal to noise ratio compared to scintillation based positron imaging. It has also high sensitivity and high spatiotemporal resolution. The spatial resolution can reach 230 μm and for positron and 55 μm for electron. The temporal resolution can reach within 0.1 second. It is also less sensitive to gamma rays.

A layer of protection foil (e.g. Aluminum, Mylar) is usually placed on top of the detector to protect the detector against scratch and to filter out visible light.

The application of this radionuclide imaging camera with silicon pixel detector was restricted with ex vivo tissue sections or easily accessible in vivo objects. It was not possible to directly extend it for the imaging on conventional transparent window chamber. First, the geometry of the silicon detector did not fit with the window geometry of the conventional transparent window chamber. Second, the silicon detector protruded only very tiny from the camera surface (<0.5 mm) and the thickness of the chamber was relatively large for the conventional window chamber (>2 mm). Thus the silicon detector of the radionuclide imaging camera cannot be inserted enough deep into a conventional window chamber to get sufficient close contact with the tissue. Without a close interaction with the imaging object, the resolution of the positron or electron imaging is very small. A 100 μm air gap will lead to a reduction of resolution for approximately 100 μm.

The apparatus of the present invention consists of three modules: 1) transparent window chamber, 2) radionuclide imaging camera and 3) supporting parts for multi-modality imaging.

The transparent window chamber in the present invention is implanted in an animal, in particular a mouse or a rat. For example, a dorsal skin window chamber can be implanted by removing one layer of skin within the observation window of one side of the chamber. A cranial window chamber can be implanted by removing the skin and bone of a region on top of the skull to generate an observation window area. Tumors can be transplanted into the remaining tissue after the window chamber implantation.

The transparent window chamber in the present invention is compatible with multimodal intravital imaging. In particular it is compatible with a direct high-quality in vivo radionuclide imaging camera, magnetic resonance imaging (MRI) and optical microscopy including fluorescence, phosphorescence, confocal or multi-photon microscopy.

The geometry of the transparent window chamber in the present invention is shaped to allow the insertion of the imaging detector of the radionuclide imaging camera, the placement of MRI surface coils and the observation using microscope. In particular the current observation window is relatively large and square-shaped, which allows the insertion of typical square-shaped detectors of radionuclide imaging cameras.

The transparent window chamber in the present invention has dedicated slots to fix the imaging parts mechanically to allow a precise co-registration of the obtained images. In particular it has fixation pins with threads to fix different imaging parts and allows direct co-registration of the obtained images.

The transparent window chamber in the present invention is fabricated with both biocompatible and MRI-compatible material to allow the application in animal and the imaging in MRI.

The thickness of the transparent window chamber in the present invention is reduced to be as less as possible to allow a close interaction between the detector and the tissue. In particular, it is less than 1.5 mm.

The slots of fixation screws of the transparent window chamber in the present invention locate on side of the transparent window chamber and the screws do not penetrate the tissues, which can minimize the invasiveness to the animal.

The transparent window chamber in the present invention can be specified as in vivo observation window on animals; in particular dorsal skin window chamber, cranial chamber, hamster-cheek-pouch-window and spine cord chamber spiral chamber.

The second part of the present invention is a direct radionuclide imaging camera, which images the signal of positron or electron exclusively with the camera itself. It does not need any functional material outside the camera to support the imaging procedure. In particular, the camera consists of a silicon-pixel detector and a single-pixel counting readout circuit.

The geometry of the radionuclide imaging camera in the present invention is shaped to fit with the geometry of the transparent window chamber. In particular, additional material is added between the detector and the camera board to allow enough protruding of the detector from the surrounding surfaces. This ensures a close contact of the detector to the investigating tissue and allows a high-resolution direct imaging of the positrons or electrons emitted by the tissue.

The third module of this apparatus is supporting parts for compatibility with multimodality imaging. This includes 1) functional supporting parts such as surface coils for MRI imaging, 2) positioning and immobilization parts such as anesthesia tube, fixation pins, fixation of surface coils and 3) other assistance parts such as magnetic shielding, fixation of glass slides, catheters, and cables.

A surface coil can be attached to the transparent window chamber to allow high-resolution MRI imaging. This surface coil assures high sensitivity and B1 homogeneity and it is fixed to the transparent window chamber mechanically.

The anesthesia tube of the present invention supports the anesthesia of the animal and the positioning of the imaging devices. The anesthesia tube consists of two parts, one base part with anesthesia gas connectors and one cover with slots hosting the placement of the transparent window chamber.

The fixation pins on the transparent window chamber of the present invention is another example of immobilization parts, which serves as the reference points for the direct co-registration of images from multiple imaging modalities.

### DESCRIPTION OF THE DRAWINGS

FIG. 1 is a sketch of an example system of the present invention. It comprises a specialized transparent window chamber, an adapted radionuclide imaging camera, glass slides to cover the tissues within the observation window, fixation for glass slides and supporting parts for multi-modality imaging such as animal anesthesia tube, MRI surface coils, fixation of surface coils and so on.

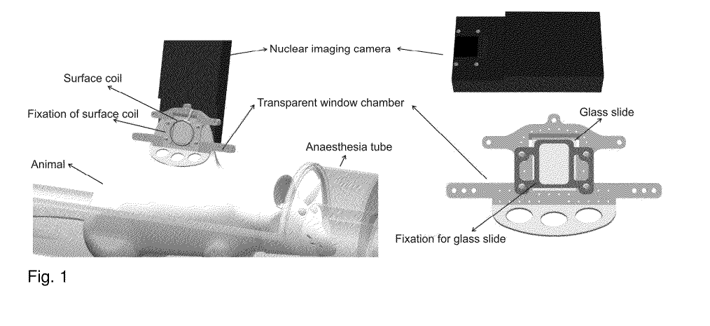

FIG. 2 shows an example of a dorsal skin transparent window chamber of the present invention. It comprises the main body 1, an observation window 2 to allow the insertion of the detector of the radionuclide imaging camera, an assisting window 3 to allow the insertion of other obligatory non-imaging parts of the radionuclide imaging camera, four fixation pins with threads 4 to allow the fixation of the transparent window chamber with other parts, a series of suture holes 5 to allow the binding of sutures with the tissues for the fixation of the transparent window chamber, screw holes 6 for the fixation of the plates of the transparent window chamber with screws, assistance excavation 7 to allow the fit with other small protruding details on the camera surface and side wing 8 to protect the dorsal skin window chamber against tilting.

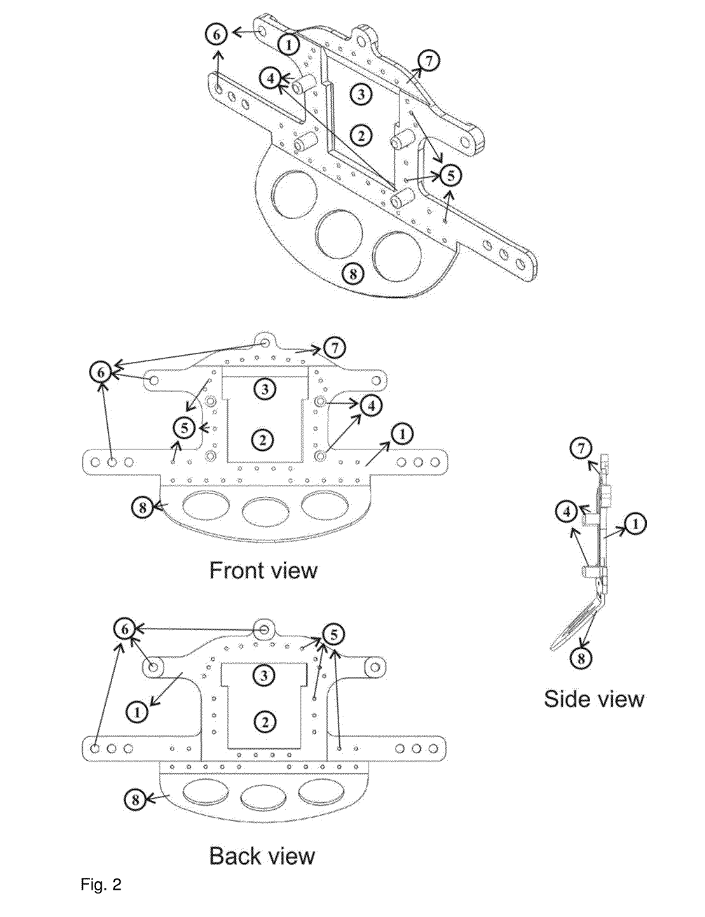

FIG. 3 shows an example of the fixation of glass slide for the transparent window chamber of the present invention. It comprises the main body of this fixation part 1, four holes 2 for the insertion of fixation pins of the transparent window chamber, protrusion 3 for the insertion into the observation window to fix the glass slide. After the chamber implantation, the subcutaneous tissue will be covered by a glass slide, which will be fixed using this part. An observation window 4 above the glass slide allows the monitoring of the tumor growth within the chamber. During the imaging procedure, the glass slide and this fixation will be detached from the chamber.

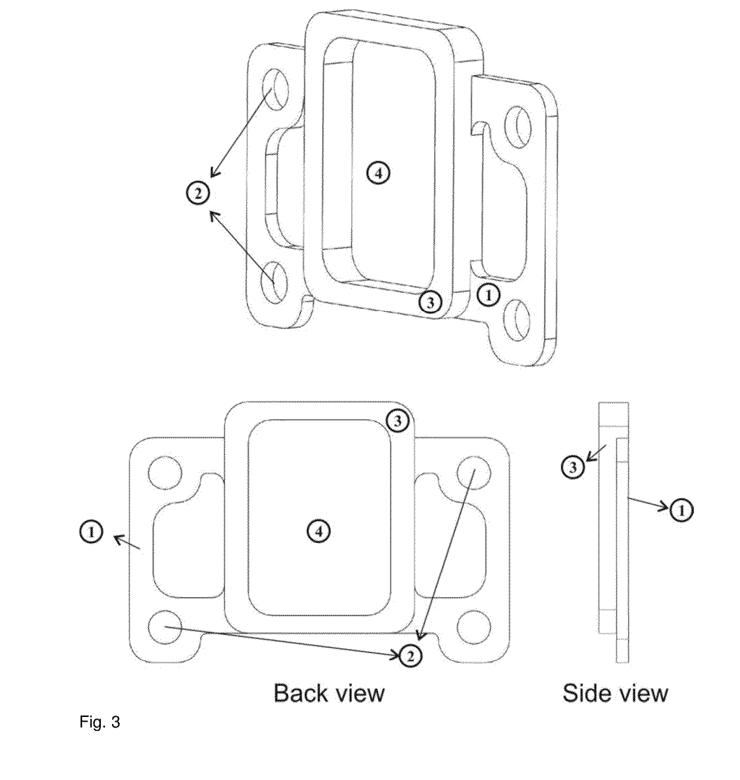

FIG. 4 shows an example of the fixation of MRI surface coil for the transparent window chamber of the present invention. It comprises the main body of this fixation part 1 with an observation window 2 in the middle, two slots 3 to host the MRI surface coil and four holes 4 for the insertion of fixation pins of the transparent window chamber.

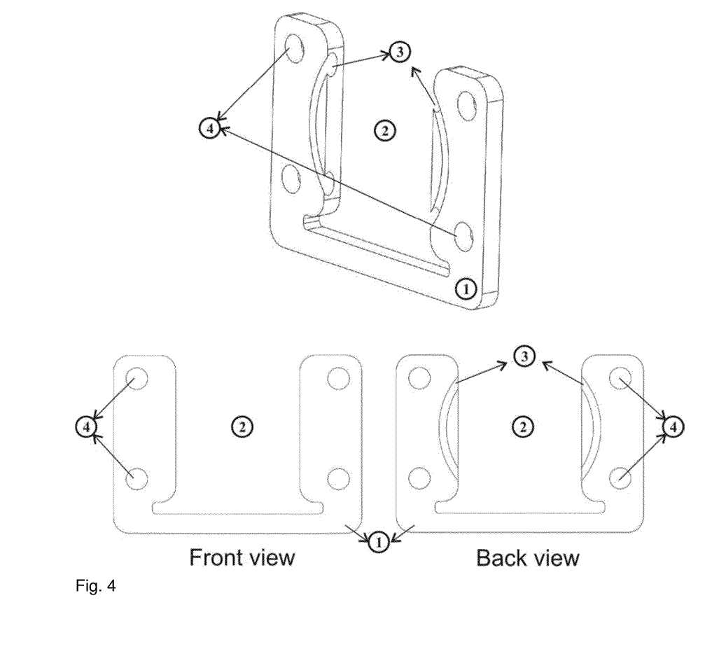

FIG. 5 shows an example of the anesthesia tube for the animal, in particular a mouse or a rat. It comprises a base 1 of the anesthesia tube and the corresponding cover 2. The base 1 is a half tube for the placement of the animal. It consists of a connector 6 for anesthesia gas including a gas inlet 7 and a gas outlet 8. The edge 4 of the cover 2 can be stuck into the slots 5 of the base 1. On the top of the cover 1 exists a slot 3 hosting the placement of the transparent window chamber in the anesthesia tube.

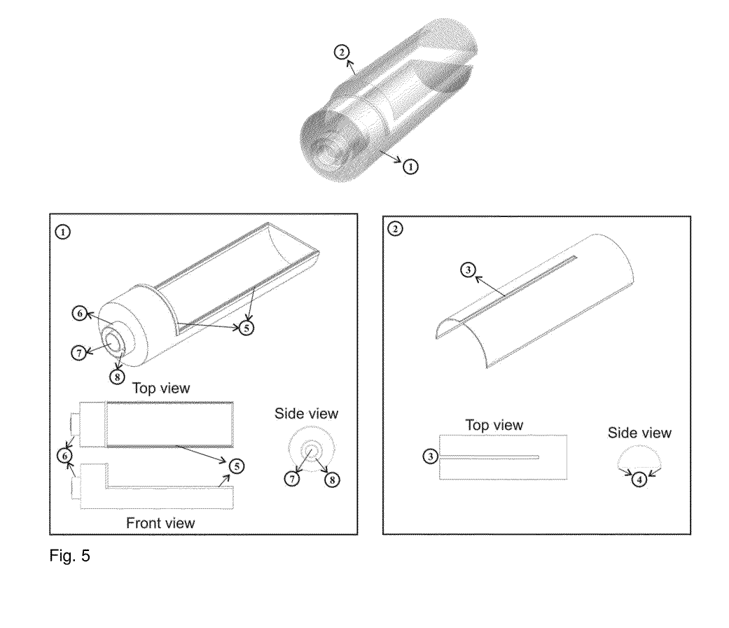

FIG. 6 shows another example of the system for cranial window chamber of the present invention. The cranial chamber 1 comprises an observation window 2 to allow the insertion of the detector of the radionuclide imaging camera, an assisting window 3 to allow the insertion of other obligatory non-imaging parts of the radionuclide imaging camera, four fixation pins with threads 4 to allow the fixation of the transparent window chamber with other parts,

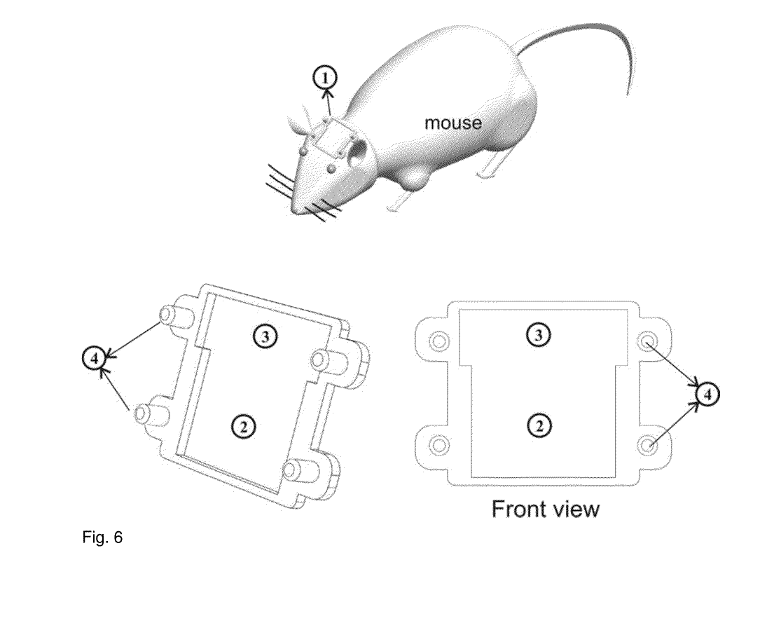

1. A transparent window chamber apparatus for multimodal intravital imaging is compatible with a direct high-quality in vivo radionuclide imaging camera, magnetic resonance imaging (MRI) and optical microscopy including fluorescence, phosphorescence, confocal or multi-photon microscopy.

2. The transparent window chamber according to claim 1 refers to an in vivo tissue observation window on animals. In particular, it refers to a dorsal skin window chamber, or a cranial chamber, or a hamster-cheek-pouch-window or a spine cord chamber.

3. The geometry of the transparent window chamber according to claim 1 and 2 is shaped to allow the insertion of the imaging detector of the radionuclide imaging camera, the placement of MRI surface coils and the positioning on a microscope. The chamber is fabricated with both biocompatible and MRI-compatible material.

4. The attachment of the transparent window chamber according to claim 1 to the tissue has minimal invasiveness. In particular, the screws for the fixation of the plates of the transparent window chamber do not penetrate the tissues.

5. The transparent window chamber according to claim 1-3 fix the imaging parts of different imaging modalities mechanically to allow direct precise co-registration of the obtained images from different sources at different times. In particular, it has fixation pins to fix the radionuclide imaging camera, MRI surface coils and microscopes.

6. The radionuclide imaging camera according to claim 1, which detects the signal of positron or electron, images the tissue object exclusively with the camera itself. It does not need any functional material between the camera and the imaging object to support the imaging procedure. The radionuclide imaging camera comprises an integrated imaging detector and a reading out circuit. In particular, the camera consists of a silicon-pixel detector and a single pixel readout circuit.

7. The geometry of the radionuclide imaging camera according to claims 1 and 6 is shaped to fit with the geometry of the transparent window chamber according to claim 1 and the imaging object. In particular, the detector of the camera is shaped to protrude from the surrounding surfaces with at least more than 1 mm distance to fit with the observation window.
